(19) United States
(12) Patent Application Publication
(10) Pub. No.: US 2007/0154398 A1
(43) Pub. Date: Jul. 5, 2007

(54) Block copolymers and nano micelles comprising the same

(75) Inventors: Chau-Hui Wang, Kaohsiung City (TW); Ging-Ho Hsiue, Hsinchu (TW); Chin-I Lin, Tainan County (TW); Shian-Jy Jassy Wang, Hsinchu County (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/714,252

(22) Filed: Mar. 6, 2007

Related U.S. Application Data
(63) Continuation-in-part of application No. 11/448,015, filed on Jun. 7, 2006.

(30) Foreign Application Priority Data
Dec. 30, 2005 (TW) 094147865

(51) Int. Cl. A61K 49/10; A61K 31/787; C08L 89/00; C08G 63/91; C08G 63/48; A61K 31/7048; A61K 31/409

(52) U.S. Cl. 424/9.34; 424/78.27; 424/78.3; 525/54.1; 525/437; 977/906; 514/185; 514/34; 514/283; 514/410

(57) ABSTRACT

A block copolymer. The block copolymer has formula A-B-C, wherein A represents polyester, B represents polyamide, and C represents specific molecular groups or metal complexes. The invention also provides a nano micelle and drug carrier including the block copolymer, wherein the drug carrier is delivered by oral, transdermal administration, injection, or inhalation.

This application is a continuation-in-part of application Ser. No. 11/448,015 filed on Jun. 7, 2006, now pending.

### BACKGROUND OF THE INVENTION

1. Field of the Invention

The invention relates to a polymer, and in particular to a block copolymer and a nano micelle and drug carrier comprising the same.

2. Description of the Related Art

Targeted drugs can be precisely focused, increasing curative effect and reducing side effect. Thus, development of drug delivery carriers having target functionality is desirable.

Recently, while pH-sensitive polymeric micelle research is popular, most deals only with physical properties they have, but rarely application for drug release. Such polymeric micelle configuration is varied due to alteration of hydrophilicity or charges of ionic-type polyelectrolyte grafted thereon. According to related reports, poly(2-ethyl-2-oxazoline) (PEOz) is a potential biomedical polymer worth developing.

### BRIEF SUMMARY OF THE INVENTION

The invention provides a block copolymer having formula A-B-C, wherein A comprises polyester, B comprises polyamide, and C comprises specific molecular groups or metal complexes.

The invention also provides a nano micelle comprising a plurality of disclosed block copolymers or a plurality of block copolymers comprising specific molecular groups and metal complexes.

The invention further provides a nano drug carrier comprising the disclosed nano micelle and a drug encapsulated thereinto.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawing, wherein:

FIG. 1 shows micelle CMC of the invention.

FIG. 2 shows polymer cytotoxicity of the invention.

FIG. 3 shows a growth inhibition assay for normal and cancer cells of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a block copolymer having formula A-B-C. The block copolymer may comprise diblock copolymer. In the formula, A may comprise polyester such as polylactide (PLA) or derivatives thereof. B may comprise polyamide such as polyoxazoline (POz) or derivatives thereof. C may comprise specific molecular groups or metal complexes. The specific molecular group, capable of recognition of cancer cells, may comprise folate or antibody. The metal complex may comprise magnetic resonance imaging (MRI) contrast agents such as chelates formed by diethylenetriaminepentaacetic acid (DTPA) and gadolinium ions (Gd³⁺) or indium (¹¹¹In), with a chelating ratio of about 1˜100 wt %.

When folate is selected as a specific molecular group, the copolymer has formula (I)

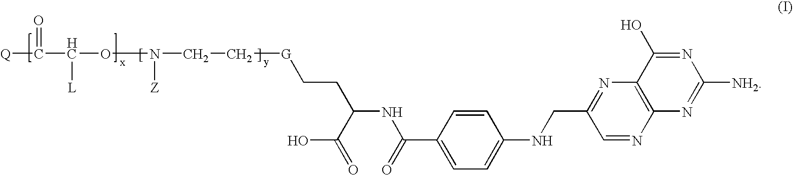

In formula (I), Q may comprise initiators for ring-opening polymerization such as hydrogen, alkyl, or hydroxyl groups, preferably hydroxyl groups. L may comprise hydrogen or C1-6 alkyl. Z may comprise hydrogen, C2-21 acyl, or C2-21 benzyl. G may comprise acyl or ester groups, preferably acyl groups. x and y may be 1˜10,000. y/x may be 0.1˜1,000.

When diethylenetriaminepentaacetic acid (DTPA) is selected as a metal complex, the copolymer has formula (II)

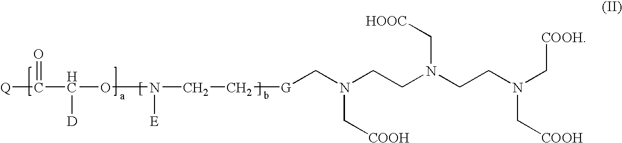

In formula (II), Q may comprise initiators for ring-opening polymerization such as hydrogen, alkyl, or hydroxyl groups, preferably hydroxyl groups. D may comprise hydrogen or C1-6 alkyl. E may comprise hydrogen, C2-21 acyl, or C2-21 benzyl. G may comprise acyl or ester groups, preferably acyl groups. a and b may be 1˜10,000. b/a may be 0.1˜1,000.

The invention also provides a nano micelle comprising a plurality of disclosed block copolymers or a plurality of block copolymers comprising specific molecular groups and metal complexes. The nano micelle have a hydrophobic interior and hydrophilic exterior, a diameter of about 10˜1,000nm, preferably 20˜200nm. The critical micelle concentration (CMC,) thereof is about 0.0001˜1 mg/mL. When MRI contrast agent is grafted thereon, a relaxivity (r₁) of about 5.0˜6.0(mM·s)⁻¹ can be achieved.

The invention further provides a nano drug carrier comprising the disclosed nano micelle and a drug encapsulated thereinto.

The drug may comprise water-insoluble drugs such as camptothecin, doxorubicin, SN-38, Paclitaxel (Taxol), Foscan-PDT (temoporfin, mTHPC), Photofrin (porfimer sodium), aminolevulinic acid (ALA), Visudyne (verteporfin), or derivatives thereof. The nano drug carrier may be delivered by oral, transdermal administration, injection, or inhalation.

The folate-grafted micelle can successfully enter tumor cells. At this time, the micelle collapses to release drugs due to (decreased pH (4˜5) of endosome caused by increased hydrogen ions. Thus, drug release can be controlled, avoiding undesirable side effects. Folate, a water-soluble small compound of vitamin B complex, is an essential substance for cell growth and differentiation. Large quantities of folate are required for tumor cell growth due to more folate receptors thereof than normal cells. Thus, the invention provides the folate-grafted micelle containing drug to kill cancer cells via receptor-mediated endocytosis.

Additionally, toxicity of gadolinium ions (Gd³⁺) or indium (¹¹¹In) can be reduced by chelating with diethylenetriaminepentaacetic acid (DTPA). The polymeric micelle also prolongs retention time of contrast agent, facilitating observation on drug distribution and accumulation to analyze patients' condition, suitable for use in magnetic resonance imaging (MRI) and single photon emission computed tomography (SPECT). Thus, the polymeric micelle can provide tumor recognition, drug release control, and molecular image exhibition simultaneously.

The invention provides two novel copolymers, such as folate-poly(2-ethyl-2-oxazoline)-block-polylactide (Folate-PEOz-b-PLA) and diethylenetriamine pentaacetic acid-poly(2-ethyl-2-oxazoline)-block-polylactide (DTPA-PEOz-b-PLA), prepared by the same template “PEOz-b-PLA”. The micelle formed by the two copolymers achieves optimal contrast and treatment effects by adjusting their composition ratios, capable of recognition of cancer cells and observation on drug release simultaneously.

The block copolymer is prepared as follows. A hydroxyl polyester such as hydroxyl polylactide (PLA-OH) is reacted with a sulfonic acid reagent such as mesyl chloride (MsCl) or toluenesulfonyl chloride (TsCl) to form a polymeric initiator such as PLA-OMs or PLA-OTs. A cationic ring-opening polymerization is initiated by adding an amide monomer such as 2-ethyl-2-oxazoline (EOz) thereto. After an ammonia acetonitrile solution is added, the polymerization is terminated and a copolymer with a terminal amino group such as PLA-b-PEOz-NH₂ is formed. Finally, a specific molecular group such as folic acid or a metal chelator such as diethylenetriamine pentaacetic acid (DTPA) monoanhydride is reacted with the amino copolymer and a block copolymer is prepared such as folate-poly(2-ethyl-2-oxazoline)-block-polylactide (Folate-PEOz-b-PLA) or diethylenetriamine pentaacetic acid-poly(2-ethyl-2-oxazoline)-block-polylactide (DTPA-PEOz-b-PLA).

### EXAMPLE 1

### Folate-poly(2-ethyl-2-oxazoline)-block-polylactide (Folate-PEOz-b-PLA) Preparation

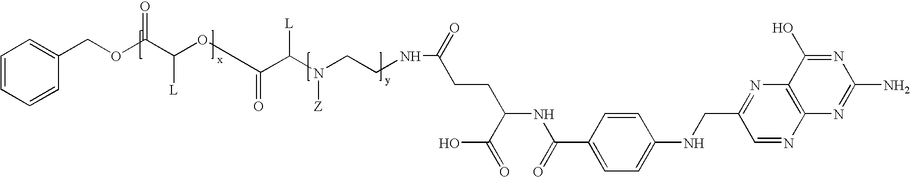

### (1) Initiator (PLA-OMs) Preparation

0.10 ml benzyl alcohol and 5 g lactide were added to a flask connected with condesing tube and vacuum tube. After nitrogen gas replacement 3 times, 15 ml dried toluene was added and heated to 140° C. with reflux. Next, 1 wt % Sn(Oct)₂ was added to react for 24 hours. After cooling, impurities were removed via celite chromatographic column with dichloromethane mobile phase. Next, the results were purified by precipitating into cooling n-hexane/ether (3/1(v/v)) solution for 2 times. The formed 4.45 g polylactide (PLA), 22 ml tetrahydrofuran, and 1.08 g triethylamine were then mixed in ice bath with stirring to form a PLA solution. Next, 22 ml tetrahydrofuran containing 1.02 g mesyl chloride was added slowly and reacted therewith for 6 hours in ice bath and 2 days under room temperature. After ultrasonic-shaking, the salt product was filtered by celite chromatographic column. Solvent was then removed. Next, the results were dissolved in 100 dichloromethane and extracted by 100 ml water, 0.1N sodium hydroxide, 0.1N hydrochloric acid, and water, respectively. After precipitating into cooling isopropyl alcohol for 2 times, the macroinitiator (PLA-OMs) was prepared.

### (2) Poly(2-ethyl-2-oxazoline)-block-polylactide (PEOz-b-PLA) Preparation

3 g polymeric initiator (PLA-OMs) was added to a flask connected with condensing tube and vacuum tube. After 3 limes replacement of dried nitrogen gas, 18 ml acetonitrile was added and reacted in oil bath at 100° C. Next, 3 ml dried 2-ethyl-2-oxazoline monomer was added to react for 48 hours with reflux. 75 ml ammonia/acetonitrile solution (0.1N) was then added and reacted for 2 hours in ice bath under nitrogen gas to conduct terminal amino group to the polymer. After dilution in acetonitrile, filtration by silica chromatographic column, and precipitation in cooling ether, the poly(2-ethyl-2-oxazoline)-block-polylactide (PEOz-b-PLA) copolymer was prepared.

### (3) Folate-poly(2-ethyl-2-oxazoline)-block-polylactide (Folate-PEOz-b-PLA) Preparation

1.13 g folate, 0.3 g N-hydroxyl succinimide, 5.16 ml triethylamine, and 0.6 g N,N′-dicyclohexylcarbodiimide (DCC) were added to 110 ml dimethyl sulfoxide (DMSO) and reacted for 4 hours at room temperature. Next, 11 g poly(2-ethyl-2-oxazoline)-block-polylactide (PEOz-b-PLA) was added and reacted overnight at room temperature. The results were then dialyzed in dimethyl sulfoxide (DMSO) and water, respectively, with dialysis film (Mw 3500) for 2 days to remove unreacted impurities. The folate-poly(2-ethyl-2-oxazoline)-block-polylactide (Folate-PEOz-b-PLA) was finally prepared.

### EXAMPLE 2

### Diethylenetriamine pentaacetic acid-poly(2-ethyl-2-oxazoline)-block-polylactide (DTPA-PEOz-b-PLA) Preparation

### (1) Initiator (PLA-OMs) Preparation

0.10 ml benzyl alcohol and 5 g lactide were added to a flask connected with condensing tube and vacuum tube. After nitrogen gas replacement 3 times, 15 ml dried toluene was added and heated to 140° C. with reflux. Next, 1 wt % Sn(Oct)₂ was added to react for 24 hours. After cooling, impurities were removed via celite chromatographic column with dichloromethane mobile phase. Next, the results were purified by precipitating into cooling n-hexane/ether (3/1(v/v)) solution for 2 times. The formed 4.45 g polylactide (PLA), 22 ml tetrahydrofuran, and 1.08 g triethylamine were then mixed in ice bath with stirring to form a PLA solution. Next, 22 ml tetrahydrofuran containing 1.02 g mesyl chloride was added slowly and reacted therewith for 6 hours in ice bath and 2 days under room temperature. After ultrasonic-shaking, the salt product was filtered by celite chromatographic column. Solvent was then removed. Next, the results were dissolved in 100 ml dichloromethane and extracted by 100 ml water, 0.1N sodium hydroxide, 0.1N hydrochloric acid, and water, respectively. After precipitating into cooling isopropyl alcohol for 2 times, the macroinitiator (PLA-OMs) was prepared.

### (2) Poly(2-ethyl-2-oxazoline)-block-polylactide (PEOz-b-PLA) Preparation

3 g polymeric initiator (PLA-OMs) was added to a flask connected with condensing tube and vacuum tube. After 3 times replacement of dried nitrogen gas, 18 ml acetonitrile was added and reacted in oil bath at 100° C. Next, 3 ml dried 2-ethyl-2-oxazoline monomer was added to react for 48 hours with reflux. 75 ml ammonia/acetonitrile solution (0.1N) was then added and reacted for 2 hours in ice bath under nitrogen gas to conduct terminal amino group to the polymer. After dilution in acetonitrile, filtration by silica chromatographic column, and precipitation in cooling ether, the poly(2-ethyl-2-oxazoline)-block-polylactide (PEOz-b-PLA) copolymer was prepared.

### (3) Diethylenetriamine pentaacetic acid-poly(2-ethyl-2-oxazoline)-block-polylactide (DTPA-PEOz-b-PLA) Preparation

3.53 g diethylenetriamine pentaacetic acid and 7 g N,N′-dicyclohexylcarbodiimide (DCC) were added to 250 ml dimethyl sulfoxide (DMSO) and reacted for 24 hours at room temperature. Next, 11 g poly(2-ethyl-2-oxazoline)-block-polylactide (PEOz-b-PLA) and 0.42 ml 1,5-diazabicyclo[4.3.0]non-5-ene (DBN) were added and reacted overnight at room temperature. The results were then dialyzed in dimethyl sulfoxide (DMSO) and water, respectively, with dialysis film (Mw 3500) for 2 days to remove unreacted impurities. The diethylenetriamine pentaacetic acid-poly(2-ethyl-2-oxazoline)-block-polylactide (DTPA-PEOz-b-PLA) was finally prepared.

### EXAMPLE 3

Micelle CMC Measurement

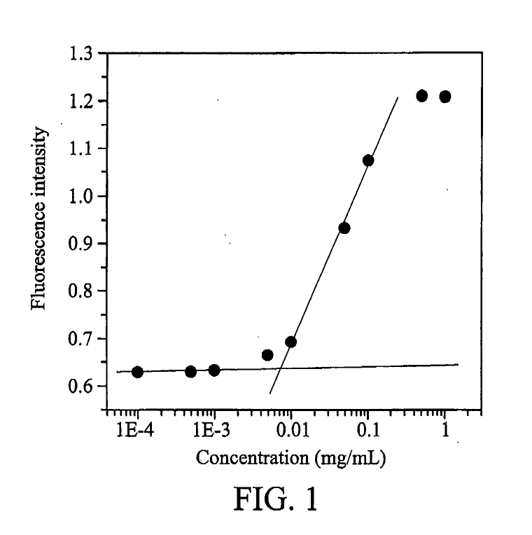

The micelle CMC was determined using a fluorescence technique. Ten microliter of acetone with pyrene (6×10⁻⁷ M) was added to 3 mL of polymer solution. This stock solution was left in the dark for 16 h. Fluorescence measurements were made using a fluorescence spectrophotometer (FluoroMax-3, Jobin Yvon). Finally, absorption and polymeric concentration logarithm were plotted. In the figure, the intersection of various slopes represents the CMC of the polymeric micelle, of 0.007 mg/mL, as shown in FIG. 1.

### EXAMPLE 4

Micelle Preparation and Size Analysis

10 mg copolymer was dissolved in 1 ml dimethyl sulfoxide (DMSO) to form a solution. The resulting solution was then dialyzed for 24 hours to form a micelle solution. Finally, micelle size and molecular weight distribution thereof were analyzed by dynamic light scattering (Malvern Instrument Zetasizer Nano ZS). The block copolymer had a molecular weight of 7800, composed of polylactide of 1000 and poly(2-ethyl-2-oxazoline) of 6800. The micelle had a diameter of 30˜31 nm.

### EXAMPLE 5

Nano Drug Carrier Preparation

8 mg folate-poly(2-ethyl-2-oxazoline)-block-polylactide (Folate-PEOz-b-PLA), 2 mg diethylenetriamine pentaacetic acid-poly(2-ethyl-2-oxazoline)-block-polylactide (DTPA-PEOz-b-PLA), and 1.5 mg camptothecin (CPT) were dissolved in 10 ml dimethyl sulfoxide (DMSO). Next, the solution was dialyzed with dialysis film (Mw3500) for 2 days. After freeze-drying, a micelle carrier packaging camptothecin of 106 nm was obtained.

### EXAMPLE 6

Micelle Cytotoxicity Assay

Human diploid fibroblast (HFW) was cultured in medium under 5% CO₂ at constant temperature of 37° C. and observed via handstand-microscope. After reaching 80% growth, cells were washed off by 0.25% trypsin-EDTA and mixed with cell stain “trypan blue”. The number of cells was then counted by cell counter. Next, 10,000 cells were planted in each pit of 96-pits culture dish. After 24 hours, the original media were replaced by flesh media containing polymers with various concentrations such as 0, 50, 100, 250, 500, and 1000 μg/ml. After 24 and 72 hours, flesh media were substituted for the polymeric media. 10 μg MTT (5 mg/ml in PBS) was then added and reacted for 4 hours. After removing the media, 100 μL dimethyl sulfoxide (DMSO) was added to dissolve crystals. After 12 hours, the absorption at 570 nm of the cells was read by 96-pits enzyme analysis instrument and repeated 6 times (n=6). Finally, the cell viability was obtained from the following formula.
cell viability (%)=absorption(sainple)/absorption(positive control)

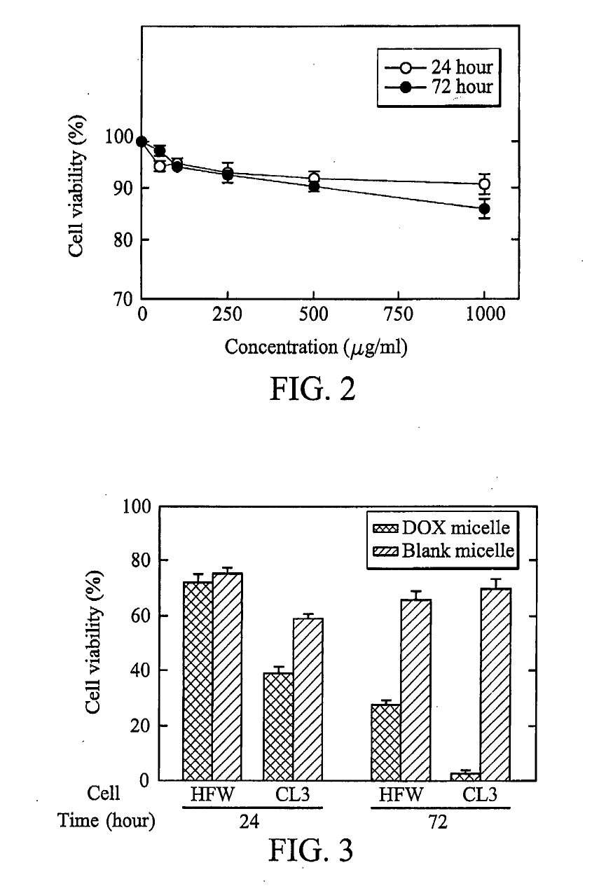

Referring to FIG. 2, the results indicate that the cell viability of the human diploid fibroblast (HFW) exceeds 80% in various polymer concentrations after culturing for 24 or 72 hours, that is, the polymeric micelle of the invention has low toxicity.

### EXAMPLE 7

Growth Inhibition Assay for Normal and Cancer Cells

Human diploid fibroblast (HFW) and lung cancer cells (CL3) were, respectively, cultured in medium under 5% CO₂ at constant temperature of 37␣ and observed via handstand-microscope. After reaching 80% growth, cells were washed off by 0.25% trypsin-EDTA and mixed with cell stain “trypan blue”. The number of cells was then counted by cell counter. Next, 10,000 cells were planted in each pit of 96-pits culture dish. After 24 hours, the original media were replaced by 10 μg/ml flesh media containing doxorubicin (DOX). After 24 and 72 hours, flesh media were substituted for the drug-contained media. 10 μg MTT (5 mg/ml in PBS) was then added and reacted for 4 hours. After removing the media, 100 μL dimethyl sulfoxide (DMSO) was added to dissolve crystals. After 12 hours, the absorption at 570 nm of the cells was read by 96-pits enzyme analysis instrument and repeated 6. times (n=6). Finally, the cell viability was obtained from the following formula. The positive control means media without drug-contained micelle.
cell viability (%)=absorption(sample)/absorption(positive control)

Referring to FIG. 3, the results indicate that the cell viability of the lung cancer cells (CL3) is less than 5% after 72 hours, that is, the drug-contained micelle of the invention can effectively kill cancer cells.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A block copolymer having formula A-B-C, wherein A comprises polyester, B comprises polyamide, and C comprises specific molecular groups or metal complexes.

2. The block copolymer as claimed in claim 1, wherein the block copolymer comprises diblock copolymer.

3. The block copolymer as claimed in claim 1, wherein the polyester comprises polylactide (PLA) or derivatives thereof.

4. The block copolymer as claimed in claim 1, wherein the polyamide comprises polyoxazoline (POz) or derivatives thereof.

5. The block copolymer as claimed in claim 1, wherein the specific molecular group recognizes cancer cells.

6. The block copolymer as claimed in claim 1, wherein the specific molecular group comprises folate.

7. The block copolymer as claimed in claim 6, wherein the block copolymer has formula (I) 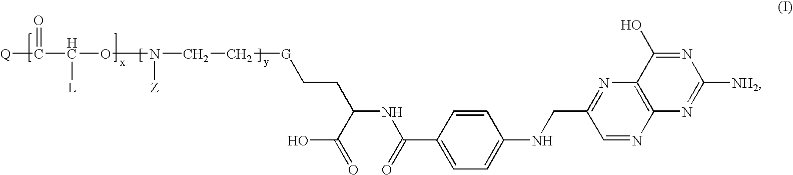
wherein L comprises hydrogen or C1-6 alkyl, Z comprises hydrogen, C2-21 acyl, or C2-21 benzyl, Q comprises initiators for ring-opening polymerization, G comprises acyl or ester groups, and x and y are 1˜10,000.

8. The block copolymer as claimed in claim 7, wherein the initiator for ring-opening polymerization comprises hydrogen, alkyl, or hydroxyl groups.

9. The block copolymer as claimed in claim 7, wherein y/x is 0.1˜1,000.

10. The block copolymer as claimed in claim 1, wherein the specific molecular group comprises antibody.

11. The block copolymer as claimed in claim 1, wherein the metal complex comprises magnetic resonance imaging (MRI) contrast agents.

12. The block copolymer as claimed in claim 11, wherein the MRI contrast agent comprises chelates formed by diethylenetriaminepentaacetic acid (DTPA) and gadolinium ions (Gd³⁺) or indium (¹¹¹In).

13. The block copolymer as claimed in claim 12, wherein the chelate formed by diethylenetriaminepentaacetic acid (DTPA) and gadolinium ions (Gd³⁺) or indium (¹¹¹In) has a chelating ratio of about 1˜100 wt %.

14. The block copolymer as claimed in claim 12, wherein the block copolymer has formula (II) 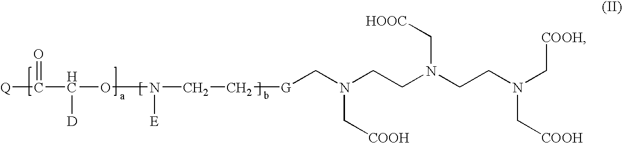
wherein D comprises hydrogen or C1-6 alkyl, E comprises hydrogen, C2-21 acyl, or C2-21 benzyl, Q comprises initiators for ring-opening polymerization, G comprises acyl or ester groups, and a and b are 1˜10,000.

15. The block copolymer as claimed in claim 14, wherein the initiator for ring-opening polymerization comprises hydrogen, alkyl, or hydroxyl groups.

16. The block copolymer as claimed in claim 14, wherein b/a is 0.1˜1,000.

17. A nano micelle comprising a plurality of block copolymers as claimed in claim 1 or a plurality of block copolymers comprising specific molecular groups and metal complexes.

18. The nano micelle as claimed in claim 17, wherein the nano micelle has a hydrophobic interior and hydrophilic exterior.

19. The nano micelle as claimed in claim 17, wherein the nano micelle has a diameter of about 10˜1,000 nm.

20. The nano micelle as claimed in claim 17, wherein the nano micelle has a diameter of about 20˜200 nm.

21. The nano micelle as claimed in claim 17, wherein the nano micelle has critical micelle concentration (CMC) of about 0.000˜1 mg/mL.

22. The nano micelle as claimed in claim 17, wherein the nano micelle has a relaxivity of about 5.0-6.0(mM·s)⁻¹.

23. A nano drug carrier, comprising:
a nano micelle as claimed in claim 17; and
a drug encapsulated thereinto.

24. The nano drug carrier as claimed in claim 23, wherein the drug comprises water-insoluble drugs.

25. The nano drug carrier as claimed in claim 23, wherein the drug comprises camptothecin, doxorubicin, SN-38, Paclitaxel (Taxol), Foscan-PDT (temoporfin, mTHPC), Photofrin (porfimer sodium), aminolevulinic acid (ALA), Visudyne (verteporfin), or derivatives thereof.

26. The nano drug carrier as claimed in claim 23, wherein the nano drug carrier is delivered by oral, transdermal administration, injection, or inhalation.
